(19) United States
(12) Patent Application Publication
(10) Pub. No.: US 2024/0156549 A1
(43) Pub. Date: May 16, 2024

(54) CAVITY MODELING SYSTEM AND CAVITY MODELING METHOD

(71) Applicant: B. Braun New Ventures GmbH, Freiburg Im Breisgau (DE)

(72) Inventor: Amir Sarvestani, Freiburg (DE)

(21) Appl. No.: 18/507,924

(22) Filed: Nov. 13, 2023

(30) Foreign Application Priority Data
Nov. 14, 2022 (DE) 10 2022 130 075.7

(51) Int. Cl. A61B 34/00; A61B 1/00; A61B 1/04; A61B 1/05; A61B 34/37; G06T 7/55; G06T 7/73; G06T 17/00; G06T 19/00

(52) U.S. Cl. CPC A61B 34/25; A61B 1/00006; A61B 1/00009; A61B 1/00045; A61B 1/00149; A61B 1/043; A61B 1/05; A61B 34/37; G06T 7/55; G06T 7/75; G06T 17/00; G06T 19/00; A61B 2034/105; G06T 2207/10064; G06T 2207/10068; G06T 2207/30016; G06T 2207/30096; G06T 2210/41; G06T 2219/004

(57) ABSTRACT

A cavity modeling method, computer-readable storage medium, computer program, and cavity modeling system can be used for intraoperative creation of a 3D model of a cavity in a patient during a surgical intervention. The modeling system includes a visualization unit with an imaging head for insertion into the patient to create an intracorporeal image of a region of the cavity. The modeling system also includes a 3D modeling unit for creating a digital 3D model of an inner surface of the cavity and augmenting and adapting it by a first image in a first image pose and by at least one second image in a second image pose. The 3D modeling unit is further adapted to output a view of the 3D model of the cavity via a visual displaying device to provide a user with a real-time intraoperative visualization of the cavity.

### CROSS-REFERENCE TO RELATED APPLICATION

This application claims priority under 35 U.S.C. § 119 to German Application No. 10 2022 130 075.7, filed on Nov. 14, 2022, the content of which is incorporated by reference herein in its entirety.

### FIELD

The present disclosure relates to a cavity modeling system/cavity scanner for an intraoperative creation of a (digital and thereby also visual or respectively visualizable) 3D model of a cavity/hollow space in a patient during a surgical intervention, in particular during a brain surgery with tumor removal, comprising: a visualization unit or a visualizing system with a distal (or respectively terminal) imaging head, which is adapted to be inserted (in particular via its configuration with corresponding geometry and dimensions) into an opening (e.g. puncture site or incision) of the patient (at least sectionally) and to create an intracorporeal image of (at least a partial portion of) a cavity of the patient via the (distal) imaging head and to make it available in digital/computer-readable form, in particular a 2D endoscope with an optical system and a downstream image sensor for creating an intracorporeal two-dimensional image. In addition, the present disclosure relates to a cavity modeling method, a computer-readable storage medium and a computer program.

### BACKGROUND

In tumor surgery in the region of the brain (neurosurgery), the main goal is to remove the tumor precisely, resecting all pathological tissue on the one hand and avoiding the removal of healthy tissue on the other hand. To this end, the tumor is often removed from the inside out, creating a hollow space/cavity in the brain. Due to the limited access, in particular for deep-seated tumors, the surgeon often does not have accurate information about the current cavity with the corresponding shape of the hollow space to compare with a preoperative plan from a magnetic resonance imaging image (MRI scan/MR scan) or a computed tomography image (CT scan) to ensure that the tumor has actually been completely removed. Such neurosurgical interventions are performed in particular with a surgical microscope, which, however, cannot detect the entire cavity, especially not the partial regions that have no line of sight or respectively no field of view for the optical axis of the surgical microscope.

In order to overcome this limitation of the view into an inner space or hollow space, surgical endoscopes are sometimes used in parallel with the surgical microscope in order to view the resection cave or hollow space from inside the patient. In this way, the surgeon can use manual guidance to gradually detect partial regions and thus the entire cavity to a certain extent (in the manner of a real-time video recording), but the individual images are only sections of the entire cavity and do not provide the surgeon with a suitable model of the cavity in order to successfully and safely perform and verify an intervention.

3D scanner or respectively 3D modeling systems for the detection of an outer surface of an object are known in dental treatment in order to create a 3D model of a tooth structure. These 3D scanners work with various technologies such as 3D laser scanners or 3D point cloud cameras. However, such scanners cannot be used in the region of neurosurgery due to the small size of the (puncture) incisions and the tumor cavity. In addition, cavities cannot be scanned and measured with such dental scanners.

### SUMMARY

The object of the present disclosure is therefore to avoid or at least reduce the disadvantages of the prior art and in particular to provide a cavity modeling system/cavity scanner, a cavity modeling method, a computer-readable storage medium and a computer program which allow a user during a surgical intervention with a visualizing system such as an endoscope, in particular a neuroendoscope, to detect a cavity/a resection cave/a resection cavity intraoperatively (i.e. during the operation) and also to create a three-dimensional model (3D model) of this cavity (at least sectionally, in particular of the entire cavity) intraoperatively on the basis of the intraoperative detection. A further partial object can be seen in offering the user an intuitive detection option with which he/she can accomplish a guided detection of the cavity/the resection cavity, in particular of the entire cavity. Another partial object is to provide a modality of an automatic or manual control in a robot-guided visualizing system or a combination of manual and automatic control, with which the user can move along the cavity and scan/detect the cavity even more easily and intuitively.

The objects of the present disclosure are solved with respect to a cavity modeling system/cavity scanner according to the present disclosure, are solved with respect to a cavity modeling method according to the present disclosure, are solved with respect to a computer-readable storage medium according to the present disclosure, and are solved with respect to a computer program according to the present disclosure.

Thus, a basic idea of the present disclosure is to provide a system for creating a three-dimensional (inner) surface model (3D model) of a hollow space which, during a surgical intervention such as a neurosurgical intervention on the brain (brain surgery) during a tumor resection, creates the 3D model, in particular using two-dimensional images (2D images) of a moving visualization unit such as an endoscope, i.e. an endoscope whose optical axis of the imaging head moves in the cavity. This makes it possible to provide a technology in which a 2D endoscope is used in particular to create a 3D model of the tumor cave in neurosurgery. In the case of neurosurgery, for example, the cavity or hollow space is located within the brain.

In particular, the cavity modeling system/cavity scanner can (may be adapted to) create a 3D model of the surgery cave using only a (conventional) 2D endoscope. For this purpose, the endoscope is inserted into the resection cave/cavity after an initial resection of the tumor. The optics of the endoscope only show a section of the cavity and only as a two-dimensional image (2D image) or respectively capture it. If the endoscope is now moved in such a way that all regions of the cavity are captured and imaged in particular, a (complete) 3D model of the resection cave can be created. The surgeon can then use this visualized 3D model to evaluate and adjust the tumor removal.

In yet other words, a cavity modeling system comprising a visualization unit (visualization system) that is (adapted to be) insertable into a surgical cavity of the patient is provided, wherein the visualizing system in particular only creates 2D images of different portions of the cavity and the cavity modeling system then creates a 3D model of the cavity based on the 2D images.

In yet other words, a cavity modeling system is provided for an intraoperative creation of a 3D (surface) model of a cavity in a patient during a surgical intervention, in particular during brain surgery with tumor removal. This cavity modeling system has a visualization unit with (a proximal handling portion for manual guidance or a connection to a robot for robotic guidance) and a distal imaging head, which is adapted to be inserted intracorporeally into the patient and to create and to digitally/computer-readably provide an intracorporeal image of at least a partial region of the cavity of the patient via the imaging head, in particular in the form of an endoscope with an optical system and a downstream image sensor for the creation of an intracorporeal image. Furthermore, the cavity modeling system has a 3D modeling unit adapted to create a digital 3D (hollow space surface) model of an inner surface of a cavity and to augment and adapt it by the provided first image in a first image pose and by at least a second image in a second image pose, wherein the 3D modeling unit is further adapted to output a view of the created 3D model of the cavity via a visual displaying device in order to provide a user, such as a medical professional, with a real-time intraoperative visualization of the cavity. The virtual 3D (hollow space surface) model is thus in particular successively augmented by the corresponding images (in the correct position) (similar to a successive panoramic image) or, respectively, if an initial 3D model is already available, adapted accordingly by the images in reference to the image poses.

The term ‘position’ means a geometric position in three-dimensional space, which is specified in particular via coordinates of a Cartesian coordinate system. In particular, the position can be specified by the three coordinates X, Y and Z.

The term ‘orientation’ in turn indicates an alignment (e.g. at the position) in space. It can also be said that the orientation indicates an alignment with a direction indication or respectively rotation indication in three-dimensional space. In particular, the orientation can be specified using three angles.

The term ‘pose’ includes both a position and an orientation. In particular, the pose can be specified using six coordinates, three position coordinates X, Y and Z and three angular coordinates for the orientation.

The term ‘3D’ defines that the image data is available spatially, i.e. three-dimensionally. The cavity of the patient or at least a partial region of the cavity with spatial extension may be digitally available in a three-dimensional space with a Cartesian coordinate system (X, Y, Z). In particular, a 3D surface model is present, i.e. in particular a closed surface in space.

The term ‘2D’ defines that the image data is available in two dimensions.

In the case of an endoscope as a visualization unit, this may in particular have angled optics, for example (an optical axis) at 60 degrees to a longitudinal axis of the endoscope. The optical axis can thus also be moved by a rotational and/or axial movement of the endoscope axis and at least a partial region, in particular the entire cavity, can be detected and recorded.

According to an embodiment, the cavity modeling system may comprise a tracking system adapted to track a position and orientation (pose) of the imaging head of the visualization unit (directly or indirectly) in space in order to determine the image pose of the image. In other words, the visualization unit/visualizing system may comprise a tracking system that is provided and adapted to determine the position and/or orientation (in particular the pose) of the visualization unit and thus the image pose in space. In particular, a transformation from a tracked handling portion to the imaging head, in particular in the form of a camera with an optical axis, can be known, so that the pose of the imaging head can be deduced from the pose of the imaging head via the detection of the pose of the handling portion and furthermore, for example via a picture analysis or via a distance sensor for three-dimensional detection of a surface, the image pose can be deduced from the pose of the imaging head and this can be determined accordingly and the image is provided together with the image pose and is augmented or adapted accordingly in the 3D model.

Preferably, the 3D modeling unit may be adapted to create a 3D model of an inner surface of the imaged region and thus of the cavity via a picture analysis of the two-dimensional image (2D scans), in which the pictures are compared for different positions. In the case of an image of the entire cavity, a complete 3D model of the cavity is created. As an alternative or in addition to picture analysis, the movement of the endoscope may also be tracked with a tracking system in order to determine the position in space for each picture and thus create a 3D model of the cavity.

According to a further embodiment, the tracking system may comprise a navigation unit with an external navigation camera and/or may comprise an electromagnetic navigation unit and/or may comprise an inertial measuring unit (inertial-based navigation unit/IMU sensor) arranged on the visualization unit, and/or the tracking system may determine the pose of the imaging head based on robot kinematics of a surgical robot with the visualization unit as end effector. In other words, the tracking/tracing of the visualization unit, in particular of the endoscope, can be realized in particular with an external optical and/or electromagnetic navigation unit and/or with an inertial-based navigation unit (internal to the visualization unit) (such as an IMU sensor in the visualization unit, in particular in the endoscope) and/or using the kinematics of a robot arm that moves the visualization unit (such as the endoscope). In particular, the position and orientation of the visualization unit can be determined by the kinematics of a robot arm that performs the movement.

Preferably, the 3D modeling unit may be adapted to determine/calculate a three-dimensional inner surface of a region of the cavity or of the entire cavity via a picture analysis based on the first image with the first image pose and the at least one second image with the second image pose. In particular, the system may thus comprise a data processing unit (such as a computer) and a storage unit as well as special algorithms for analyzing the images/pictures from different positions in order to create a 3D surface of either a region of the hollow space or of the entire hollow space.

In particular, the visualization unit, in particular the endoscope, may comprises a fluorescence imaging unit with a spotlight of a predefined wavelength for excitation and with a sensor for detection, wherein in particular fluorescence is detectable via the imaging head with image sensor in order to augment the 3D model of the inner surface of the cavity with further annotations, in particular annotations on tumor activity and/or blood flow, and to provide real-time information relevant for the intervention. In other words, the visualization unit may be, in particular, an endoscope with integrated fluorescence imaging, which supplements the 3D model (the 3D hollow space surface) with functional information, in particular with annotations on tumor activity and/or blood flow, in order to provide the user with further, real-time information relevant to the intervention.

According to one embodiment, preoperative three-dimensional images may be stored in a storage unit of the cavity modeling system, in particular MRI images and/or CT images, which comprise at least the intervention region with a tissue to be resected, in particular a tumor, and the cavity modeling system may further comprise a comparison unit adapted to compare the intraoperatively created 3D model of the inner surface of the cavity with a three-dimensional outer-surface model of the tissue to be removed, in particular tumors, of the preoperative image, and to output the comparison via the displaying device, in particular to output a deviation, particularly preferably in the form of a superimposed representation of the intraoperative 3D model and the preoperative three-dimensional surface model and/or an indication of a percentage deviation of a volume, so that regions of under-resection or over-resection are shown to the user. In particular, the cavity modeling system may thus be adapted to compare the 3D model, in particular the 3D surface of the hollow space, with the 3D (surface) model of the tumor from a preoperative 3D image such as a magnetic resonance imaging (MRI) image or computed tomography (CT) image. Furthermore, the system may preferably indicate, in particular display, deviations between the intraoperatively determined 3D model of the cavity and the 3D model of the tumor from a preoperative image such as MR or CT, so that the user can recognize regions with under-resection or over-resection.

Further preferably, the comparison unit may be adapted to compare a three-dimensional shape of the intraoperative 3D model and of the preoperative three-dimensional surface model, in particular to compare a ratio of a width to a length to a height and to output this via the displaying device, in particular with respect to a deviation, in order to illustrate the resection via the shape comparison in a cavity changing due to soft tissue and in particular to confirm a correct resection.

Preferably, the cavity modeling system may display a view of the 3D model with detected regions with the intraoperative images via the displaying device in real time and can also display the regions that have not yet been detected, and during manual guidance of the visualization unit, in particular the endoscope, can provide the user with instructions for detecting the regions that are still to be detected, in particular in the form of arrows that specify a direction of translation and/or direction of rotation in order to provide the user with an intuitive, complete detection of the cavity.

Preferably, a movement of the visualization unit may be performed either manually by a surgeon or automatically by a robot arm or by a combination of both. In particular, the visualization unit may be moved manually or by a robot or by a combination of both. For example, the visualizing system may only be moved manually by a user in order to detect/sample the surface of the hollow space. The visualization unit may also be moved in particular by a robot or respectively via a robot. This robot may in particular be completely manually controlled by the user or may move autonomously (for example according to an automatic control method for detecting the cavity) in order to scan the surface of the hollow space.

Preferably, the cavity modeling system may comprise a robot with a robot arm to which the visualization unit, in particular the endoscope, is connected as an end effector, wherein a control unit of the cavity modeling system is adapted to control the robot and thus the pose of the imaging head of the visualization unit and in particular to automatically scan the cavity for detection of the cavity, in particular in order to detect the entire inner surface of the cavity.

According to one embodiment, the control unit may be adapted to control the position of the imaging head with three position parameters and the orientation of the imaging head with three orientation parameters, wherein a subset of the (control) parameters is assigned to the automatic control and is automatically executed by the control unit and a remaining subset of the parameters is assigned to the manual control and can be controlled by the user via an input unit, wherein preferably a rotation of the imaging head is assigned to the manual control via the orientation parameters and an axial, translational movement is assigned to automatic control via the position parameters.

The visualization unit may preferably also be moved in a manual-automatic control, in which the visualization unit is on the one hand moved manually by the user, in particular only the rotation is controlled manually by the user, combined with an autonomous movement by a robot, in particular only the axial movement. For example, the rotation can be controlled manually by the user, while the robot autonomously performs the axial movement automatically, so that a combined movement of the visualization unit takes place, with both a manual control of parameters of a rotation and an automatic control of parameters of a translation or respectively an axial movement. In other words, the parameters of degrees of freedom can be divided, while a subset of the parameters is assigned to the manual control and another part of the parameters is assigned to the automatic control.

In one embodiment, automatic control can initially be carried out by the robot and, if required, manual control can replace the automatic control, partly with regard to a predefined subset of parameters of degrees of freedom or even completely, by manual control, so that, if required, automatic movement though the cavity can be overwritten by a manual movement and thus can be taken over.

In particular, the visualization unit may be configured in the form of an endoscope, in particular a 2D endoscope or a 3D endoscope, and may have a camera. In particular, its optical axis may be aligned transversely to a longitudinal axis of the endoscope, in particular the optical axis may protrude into a radial outer side of the endoscope shaft, and the endoscope may further preferably have a wide-angle camera on a radial outer side, which detects a viewing angle of over 60° in order to detect the inner surface of the cavity via rotation. The visualization unit may therefore be configured in particular in the form of a 2D endoscope, which creates two-dimensional images (2D scans). Alternatively, the visualization unit may be a 3D endoscope, which creates three-dimensional images (3D scans).

The cavity modeling system may preferably comprise a display or 3D glasses (virtual or augmented) for outputting the 2D picture to the user during the movement of the visualizing system and the generated 3D surface.

Preferably, the 3D (hollow space) model may be used to intraoperatively perform a brain shift correction when using a navigation unit by correcting the preoperative 3D image with the 3D model.

In particular, the system may be adapted to augment the 3D model with additional information from other modalities, in particular neuro-monitoring and/or histology.

In particular, the cavity modeling system may be adapted to output a percentage indication of a (spherical) detection of the cavity via the displaying device and to output a visual indication of regions of the cavity that are yet to be detected.

In particular, the visualization unit may be a rigid endoscope. In particular, the visualization unit may be a neuroendoscope.

In particular, a diameter of an endoscope shaft may be less than 10 mm, preferably less than 5 mm. In particular, a dimension of an imaging head may be less than 5 mm.

In particular, the cavity modeling system may be adapted to create the 3D model from moving images using a panorama function.

In particular, the cavity modeling system may be adapted to check the completeness of a detection of the resection cavity (cave) and, in the event that a complete detection is not yet available, issue an instruction to a user to move the visualization unit and, in the event that a complete detection is available, may issue a view of the 3D model or a comparison with a preoperative three-dimensional surface model. What is important here is a completeness feature of the detection of the inner surface of the cavity, which the cavity modeling system can determine.

In particular, tracking/tracing for a rigid endoscope can be performed indirectly via a handpiece or attached tracker (with markers) and a known rigid transformation from handpiece or tracker to imaging head.

In particular, the displaying device may be used to display regions that have been detected and also regions that have not been detected. Preferably, the cavity modeling system can guide the visualization unit to the regions that have not yet been detected.

In particular, the 3D model may be adapted to an inner surface of a hollow space. In particular, a spherical surface model may first be selected as the initial model, which is then adapted accordingly by the images around the surface in this region. In particular, a 3D surface model (with a full sleeve) can be adapted accordingly using the two-dimensional images.

With regard to a cavity modeling method for intraoperative creation of a 3D model of a cavity in a patient during a surgical intervention, in particular during brain surgery with tumor removal, the objects are solved by the cavity modeling method comprising the steps of: preferably intracorporeally inserting a visualization unit with a distal imaging head into the patient, in particular an endoscope with an optical system and a downstream image sensor for creating an intracorporeal image; creating a first image by the visualizing system in a first image pose; creating at least a second image by the visualizing system in a second image pose which is different from the first image pose; creating a 3D (hollow space surface) model of an inner surface of a cavity and augmenting and adapting by the provided first image in a first image pose and by at least a second image in a second image pose; and outputting a view of the created 3D model of the cavity via a visual displaying device in order to provide a user, such as a medical professional, with a real-time intraoperative visualization of the cavity.

In particular, the cavity modeling method may further comprise the steps of: comparing a shape of a preoperative three-dimensional surface model with the 3D model; and outputting, by the displaying device, a superimposed representation, in particular a respective partially transparent view, of the intraoperative 3D model of the inner surface of the cavity and of the preoperative three-dimensional surface model in order to visualize a resection to the user.

With respect to a computer-readable storage medium and a computer program, the objects are solved by comprising commands which, when executed by a computer, cause the computer to perform the method steps of the cavity modeling method according to the present disclosure.

Any disclosure in connection with the cavity modeling system according to the present disclosure applies as well (analogously) to the cavity modeling method according to the present disclosure and vice versa.

### BRIEF DESCRIPTION OF THE DRAWINGS

The present disclosure is explained in more detail below based on preferred embodiments with reference to the accompanying Figures.

FIG. 1 shows a perspective view of a cavity modeling system according to a first preferred embodiment of the present disclosure,

FIG. 2 shows a schematic longitudinal sectional view through a patient's brain with a cavity into which an endoscope is inserted in order to detect the cavity and create a 3D model;

FIG. 3 shows a perspective view of a further embodiment of a cavity modeling system according to a further preferred embodiment, in which the endoscope is automatically guided by a robot to detect the cavity;

FIG. 4 shows a schematic representation of image processing to illustrate how a 3D model can be created using the plurality of 2D images; and

FIG. 5 shows a flowchart of a cavity modeling method according to a first preferred embodiment.

The Figures are schematic in nature and are intended only to aid understanding of the present disclosure. Identical elements are marked with the same reference signs. The features of the various configuration examples can be interchanged.

### DETAILED DESCRIPTION

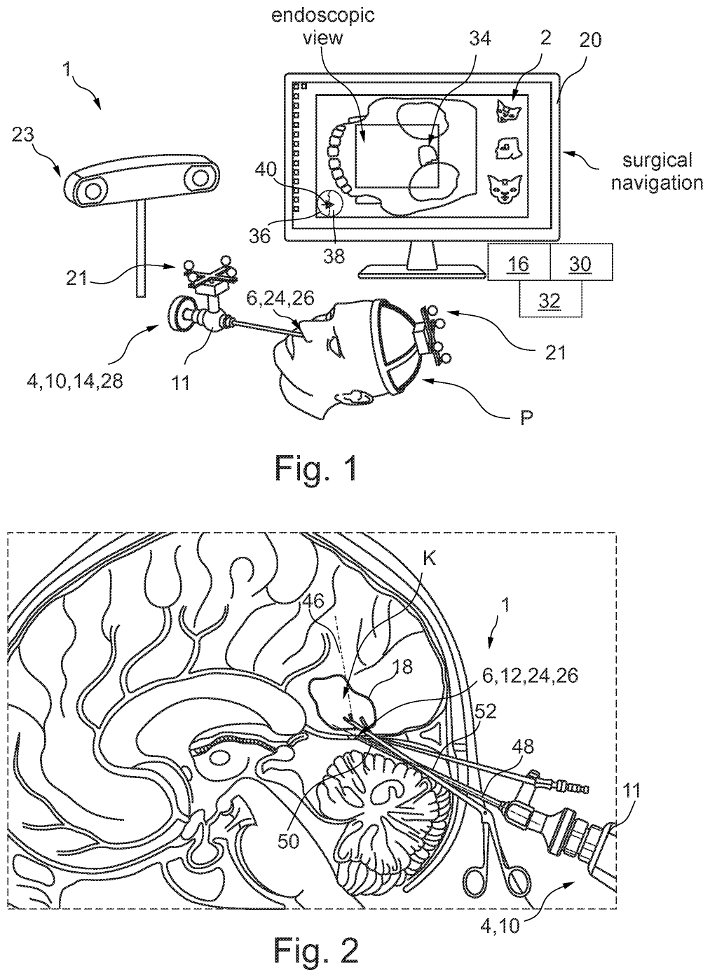

FIG. 1 shows a schematic perspective view of a cavity modeling system 1 according to a first preferred embodiment of the present disclosure, which is used in a neurosurgical intervention of a tumor removal on the brain of a patient P.

The cavity modeling system 1 comprises a visualization unit 4 with a distal imaging head 6 in the form of an endoscope 10 with a distal optical system 12 and a downstream image sensor 14. The endoscope is adapted to be inserted intracorporeally into the patient P in the brain itself and then to create and digitally provide an intracorporeal image 8 of at least a partial region of the cavity K of the patient P via the imaging head 6. The present cavity modeling system 1 has a 3D modeling unit 16 (with a processor and a memory), which is adapted to create a digital 3D model 2 of an inner surface 18 of the cavity K and to augment and adapt this with a provided first image 8a in a first image pose and with at least a second image 8b in a second image pose. The endoscope 10 may either be guided manually via its proximal handling portion 11 or may be connected to a robot in order to be moved inside the resection cave or, respectively, in the cavity K of the tumor to be removed. In this embodiment, the endoscope 10 is even moved continuously until images 8 of the entire cavity K are available, which are integrated into the 3D model 2 in order to create a complete 3D model 2 of the inner surface of the cavity K.

The images 8 are analyzed with regard to a three-dimensional inner surface of the cavity K and the 3D model 2 is created or respectively adapted at the corresponding regions with regard to the calculated three-dimensional shape. In addition to the geometric shape of the 3D model 2, the (colored) images are also included so that the image information is included in the 3D model 2 in addition to the spatial information.

The 3D modeling unit 16 is also adapted to output a view of the created 3D model 2 of the cavity K via a visual displaying device 20, in the form of a surgical monitor, in order to provide a user, such as a medical professional, with a real-time intraoperative visualization of the cavity K. Similar to a CAD model, which is rotatable around different axes and movable in space, with optional possibilities of longitudinal sections or cross sections as well as zoom functions to generate the best possible views, a view of the digital 3D model 2 can be output via the displaying device, which the surgeon uses for his/her intervention.

The cavity modeling system 1 or respectively the cavity scanner 1 thus scans a cavity K completely with the endoscope 10 (in contrast to a dental 3D scanner, for example, which is adapted to detect an object with an outer surface) and creates a digital surface model of the scanned hollow space, which contains information on a geometric shape. With the help of this 3D model 2, the surgeon can then recognize whether his resection is correct or whether there is an over-resection or under-resection.

Furthermore, the cavity modeling system 1 has a tracking system 22 that is adapted to track a position and orientation of the distal imaging head 6 of the endoscope 10 in space in order to determine the image pose of the intracorporeal image 8. In this embodiment, the tracking system is in the form of an optical navigation unit with external trackers 21 in the form of rigid bodies with optical markers and an external navigation camera 23 (in the form of a stereoscopic camera). A tracker 21 is attached to the handling portion 11 of the endoscope 10 and a rigid transformation from pose tracker to pose front lens is also known to the navigation unit, so that the image pose can be determined via this. A tracker 21 is furthermore attached to the head of the patient P so that the head and thus the intervention region with the cavity K can be tracked by the external navigation camera 23.

Furthermore, the endoscope has a fluorescence imaging unit 24 at its distal end with both a spotlight 26 (here UV spotlight) of a predefined wavelength (UV wavelength) for excitation. The image sensor 14 of the endoscope 10 serves as the sensor 28 for detecting the fluorescence, since the fluorescence is again in the wave range of visible light. In this way, annotations on tumor activity and blood flow can be added to the 3D model in order to provide the surgeon with real-time information relevant to the intervention.

Preoperative three-dimensional images are stored in a storage unit 30 of the cavity modeling system 1, in the present case MRI images which also comprise at least the intervention region with a tumor to be resected. Furthermore, the cavity modeling system 1 comprises a comparison unit 32, which is adapted to compare the intraoperatively created 3D model 2 of the inner surface 18 of the cavity K with a three-dimensional outer-surface model 34 of the tumor to be removed from the preoperative image. An intracorporeal inner surface model is thus compared with a preoperative outer surface model via the comparison unit 32 and a view of this comparison is then output via the displaying device (20). In the present case, a superimposed representation of the intraoperative 3D model 2 and the preoperative three-dimensional surface model 34 is output in order to show the surgeon the regions of the under-resection or respectively over-resection.

The cavity modeling system 1 can also display a real-time view of the 3D model 2 with the already detected regions 36 with the intraoperative images 8, 8a, 8b via the displaying device 20 and can also display the remaining, not yet detected (surface) regions 38 that have still to be detected for a complete scan of the cavity K. During manual guidance of the endoscope 10, the displaying device 20 also provides the surgeon with instructions for detecting the regions still to be detected in the form of arrows 40, which indicate a direction of translation in the form of straight arrows and a direction of rotation in the form of rotating arrows, similar to a navigation unit in a car, in order to provide the surgeon with an intuitive, complete detection modality for cavity K.

FIG. 2 shows a detailed longitudinal sectional view through a brain of the patient P, wherein a 2D endoscope 10 of a cavity modeling system 1 of a further preferred embodiment is inserted into a cavity in order to create the intracorporeal images 8 and then to generate the 3D model 2 from these images. It is easy to see that the rigid endoscope 10 can gradually detect the entire cavity through movements in the axial direction as well as rotations, and that the 3D modeling unit 16 can finally reproduce the entire cavity K in the form of the 3D model 2 on the basis of the gradually added images, and all of this intraoperatively. This allows the surgeon to check his/her success directly in the operating room.

The endoscope 10 may be configured as a 2D endoscope with a two-dimensional image 8 or as a 3D endoscope with a three-dimensional image 8. The optical axis 46 extends obliquely, in particular transversely to a longitudinal axis 48 of the endoscope 10 aligned at an angle of 60° to the longitudinal axis 48 of the endoscope 10. Thus, the optical axis 46 extends into a radial outer side or respectively outer surface 50 of an endoscope shaft 52, In particular, the endoscope 10 may have a wide-angle camera on its radial outer side 50, which captures a viewing angle of more than 60° in order to detect the inner surface 18 of the cavity K via rotation.

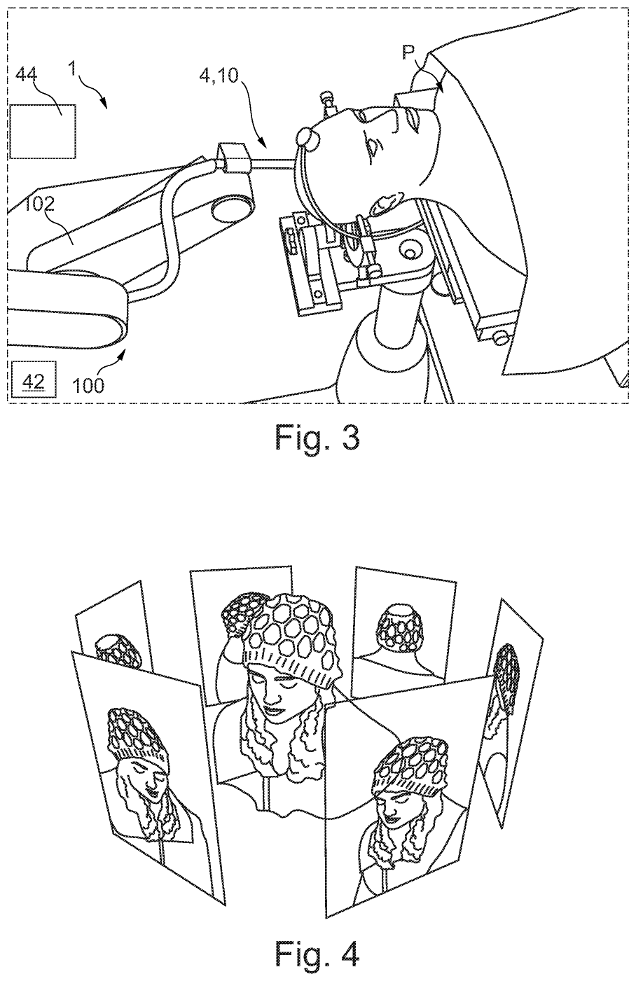

FIG. 3 shows, in contrast to the first embodiment, a robot-assisted cavity modeling system 1 according to a further, third preferred embodiment of the present disclosure. Here, the visualization unit 4 in the form of the rigid endoscope 10 is only guided by a robot 100 through its robot arm 102. The endoscope 10 is connected to the robot arm 102 as an end effector and can be moved in space. In particular, a position and orientation of the distal imaging head 6 can thus be controlled. A control unit 42 of the cavity modeling system 1 is adapted to control the robot 100 and thus the pose of the imaging head 6 and to automatically move along the cavity K for detection in order to detect the entire inner surface 18 of the cavity K. In a control modality that can be selected by the surgeon, the control unit 42 is adapted to control the position of the imaging head 6 via three position parameters and the orientation of the imaging head 6 via three orientation parameters. A subset of the parameters, namely the orientation parameters for rotation of the imaging head 6, is assigned to manual control and an axial, translational movement is assigned to automatic control via the position parameters. The user can then control the parameters of a rotation via an input unit 44, in this case a touch display.

FIG. 4 schematically shows the process for creating a 3D model based on 2D images, which can be applied analogously to a cavity. Specifically, two-dimensional images of the object are created in different directions, from which the 3D model can then be recalculated.

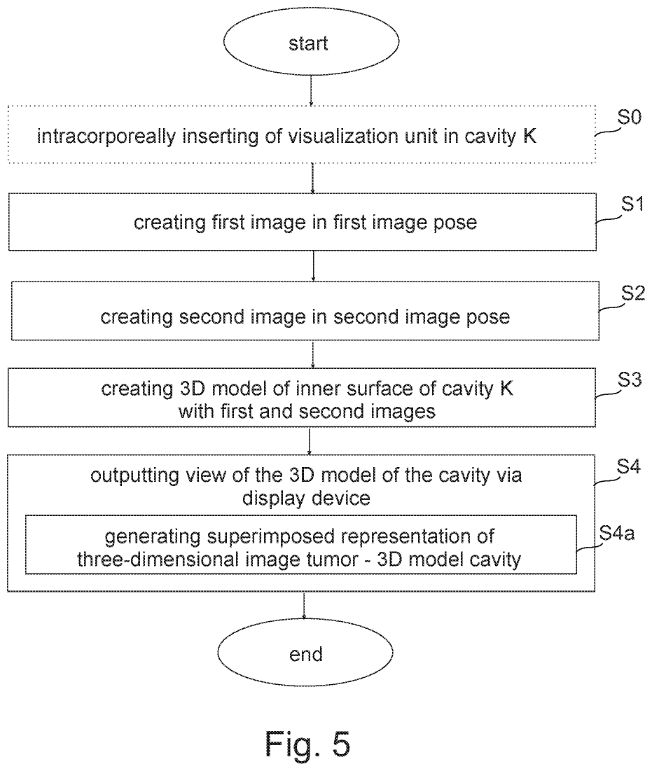

FIG. 5 shows a cavity modeling method according to a preferred embodiment. In an optional step S0, an intracorporeal insertion of a visualization unit 4 with a terminal imaging head 6 in the form of an endoscope 10 with a distal optical system 12 and a downstream image sensor 14 into a cavity K of the patient P is performed for the creation of an intracorporeal image 8 of a partial region of the cavity K.

In a first step S1, a first image 8a is created by the visualizing system 4 in a first image pose.

In a second step S2, at least one second image 8b, in the present case even a plurality of further images 8b, is created by the visualizing system 4 in a second image pose that is different from the first image pose and is provided digitally.

In step S3, a 3D model 2 of an inner surface 18 of the cavity K is created with augmentation and adaptation by the provided first image 8a in the first image pose and by at least the further images 8b in the further image poses.

Finally, in step S4, a view of the created 3D model 2 of the cavity K is output via a visual displaying device 20 in the form of a display in order to provide a medical professional such as a surgeon with a real-time intraoperative visualization of the cavity K.

1. A cavity modeling system for an intraoperative creation of a 3D model of a cavity in a patient during a surgical intervention, the cavity modeling system comprising:
a visualization unit with a distal imaging head, which is adapted to be inserted intracorporeally into the patient and to create and to digitally provide an intracorporeal image of at least a partial region of the cavity of the patient via the distal imaging head; and
a 3D modeling unit adapted to create a digital 3D model of an inner surface of the cavity and to augment and adapt the digital 3D model by a first image in a first image pose and by at least one second image in a second image pose,
the 3D modeling unit being further adapted to output a view of the digital 3D model of the cavity via a visual displaying device to provide a user with a real-time intraoperative visualization of the cavity.

2. The cavity modeling system according to claim 1, further comprising a tracking system adapted to track a position and an orientation of the distal imaging head of the visualization unit in space in order to determine the image pose of the intracorporeal image.

3. The cavity modeling system according to claim 2, wherein the tracking system at least one of:
comprises a navigation unit with an external navigation camera;
comprises an electromagnetic navigation unit;
comprises an inertial measuring unit arranged on the visualization unit;
determines a position of the imaging head based on robot kinematics of a surgical robot with the visualization unit as an end effector.

4. The cavity modeling system according to claim 1, wherein the 3D modeling unit is adapted to calculate a three-dimensional inner surface of a region of the cavity or of an entirety of the cavity via a picture analysis based on the first image in the first image pose and the at least second image in the second image pose.

5. The cavity modeling system according to claim 1, wherein the visualization unit comprises a fluorescence imaging unit with a spotlight of a predefined wavelength for excitation and with a sensor.

6. The cavity modeling system according to claim 1, wherein preoperative three-dimensional images are stored in a storage unit of the cavity modeling system, the preoperative three-dimensional images comprising at least an intervention region with a tissue to be resected, and the cavity modeling system further comprising a comparison unit adapted to compare the digital 3D model of the inner surface of the cavity with a three-dimensional outer-surface model of a tissue to be resected of the preoperative image, and to output a comparison via the visual displaying device, so that regions of under- or over-resection are indicated to the user.

7. The cavity modeling system according to claim 6, wherein the comparison unit is adapted to compare a three-dimensional shape of the intraoperative 3D model and of the preoperative three-dimensional surface model, in order to illustrate a resection via a shape comparison in a cavity changing due to soft tissue.

8. The cavity modeling system according to claim 1, wherein the cavity modeling system displays a view of the digital 3D model with detected regions with the intraoperative images via the displaying device in real time and with the remaining regions that have not yet been detected, and during manual guidance of the visualization unit, provides the user with instructions for detecting regions still to be detected, in order to provide the user with an intuitive, complete detection of the cavity.

9. The cavity modeling system according to claim 1, further comprising a robot with a robot arm to which the visualization unit is connected as an end effector, wherein a control unit of the cavity modeling system is adapted to control the robot and thus a position of the imaging head of the visualization unit.

10. The cavity modeling system according to claim 9, wherein the control unit is adapted to control the position of the imaging head via three position parameters and an orientation of the imaging head via three orientation parameters, wherein a first subset of the position parameters and the orientation parameters is assigned to an automatic control and is automatically executed by the control unit and a second subset of the position parameters and the orientation parameters is assigned to a manual control and is controllable by the user via an input unit.

11. The cavity modeling system according to claim 1, wherein the visualization unit is configured in the form of an endoscope and has a camera.

12. A cavity modeling method for an intraoperative creation of a 3D model of a cavity in a patient during a surgical intervention, the cavity modeling method comprising the steps of:
creating a first in a first image pose;
creating at least a second image in a second image pose that is different from the first image pose;
creating a 3D model of an inner surface of the cavity with augmenting and adapting by the first image in the first image pose and by the second image in the second image pose; and
outputting a view of the 3D model of the cavity via a visual displaying device in order to provide a user with a real-time intraoperative visualization of the cavity.

13. The cavity modeling method according to claim 12, further comprising the steps of:
comparing a shape of a preoperative three-dimensional surface model with the 3D model; and
outputting, by the displaying device, a superimposed representation of the 3D model of the inner surface of the cavity and of the preoperative three-dimensional surface model to visualize a resection to the user.

14. A computer-readable storage medium comprising commands which, when executed by a computer, cause the computer to perform the method steps of the cavity modeling method according to claim 12.

15. A computer program comprising commands which, when executed by a computer, cause the computer to perform the method steps of the cavity modeling method according to claim 12.
